(19) United States
(12) Patent Application Publication
(10) Pub. No.: US 2011/0177039 A1
(43) Pub. Date: Jul. 21, 2011

(54) ADULT BONE MARROW CELL TRANSPLANTATION TO TESTES CREATION OF TRANSDIFFERENTIATED TESTES GERM CELLS, LEYDIG CELLS AND SERTOLI CELLS

(75) Inventors: Yanhe Lue, Torrance, CA (US); Ronald S. Swerdloff, Long Beach, CA (US); Peter Yiwen Liu, Parramatta (AU); Krista Erkkila, Espoo (FI); Christina Wang, Long Beach, CA (US)

(73) Assignee: Los Angeles Biomedical Research Institute at Harbor-UCLA Medical Center, Torrance, CA (US)

(21) Appl. No.: 13/009,717

(22) Filed: Jan. 19, 2011

Related U.S. Application Data
(63) Continuation of application No. 11/696,314, filed on Apr. 4, 2007.
(60) Provisional application No. 60/790,085, filed on Apr. 7, 2006.

(51) Int. Cl. A61K 35/12; A61P 15/08; A61P 5/26

(52) U.S. Cl. 424/93.7

(57) ABSTRACT

This invention pertains to the discovery that stem cells (e.g., bone marrow stem cells) transplanted directly into a testicular environment are transdifferentiated into bona fide Sertoli cells, and/or Leydig cells, and/or and germ cells. This provides a mechanism for the treatment of male infertility and/or testosterone deficiency. Thus, in one embodiment, this invention provides a method of treating infertility or testosterone deficiency in a male mammal. The method typically involves implanting stem cells into the testes of the mammal whereby the stem cells differentiate into germ cells and/or Sertoli cells and/or Leydig cells thereby reducing infertility and/or testosterone deficiency.

### CROSS-REFERENCE TO RELATED APPLICATIONS

This application is a Continuation of U.S. Ser. No. 11/696,314, filed on Apr. 4, 2007, which claims benefit of and priority to U.S. Ser. No. 60/790,085, filed on Apr. 7, 2006, both of which are incorporated herein by reference in their entirety for all purposes.

### STATEMENT AS TO RIGHTS TO INVENTIONS MADE UNDER FEDERALLY SPONSORED RESEARCH AND DEVELOPMENT

[Not Applicable]

### FIELD OF THE INVENTION

This invention pertains to the field of infertility. In particular this invention pertains to the administration of stems cells to the testes to produce germ cells, Leydig cells or Sertoli cells.

### BACKGROUND OF THE INVENTION

Stem cells are undifferentiated cells defined by their ability at the single cell level to both self-renew and differentiate to produce mature progeny cells, including both non renewing progenitors and terminally differentiated effect cells (Wagers and Weissman (2004) Cell 116: 639-648). There are two kinds of stem cells: embryonic stem cells and adult stem cells, which not only have similarities but also have different functions and characteristics (Weissberg and Qasim (2005) Heart 91: 696-702). The embryonic stem (ES) cell is defined by its origin—that is from one of the earliest stages of the development of the embryo, called the blastocyst. Specifically, embryonic stem cells are derived from the inner cell mass of the blastocyst at a stage before it would implant in the uterine wall (Thomson et al. (1998) Science 282: 1145-1147). The embryonic stem cell can self-replicate and is pluripotent, it can give rise to cells derived from all three germ layers-endoderm, mesoderm, and ectoderm (Slack (2000) Science 287:1431-1433). Although ES cells have been isolated from human (Thomson et al. (1998) supra.), their use in research as well as therapeutics is encumbered by ethical considerations (Frankel (2000) Science 298: 1397; Weissman (2005) Nature (Published online 16 Oct. 2005)). The adult stem cell is an undifferentiated cell that is found in a differentiated tissues; it can renew itself and become specialized to yield all of the specialized cell types of the tissue for which it originated. Adult stem cells are capable of self-renewal for the lifetime of the organism. Stem cells also exist for most tissues, including hematopoietic (Weissman (2000) Cell 100: 157-158), neural (Gage (2000) Science 287: 1433-1438), gastrointestinal (Potten (1998) Phil. Trans. R. Soc. Lond. B 353: 821-830), epidermal (Watt (1997) Phil. Trans. R. Soc. Lond 353: 831), hepatic (Alison and Sarraf (1998) J Hepatol., 29: 678-683) and mesenchymal stem cells (Pittenger et al. (1999) Science 279: 528-530), as well as ovary (Johnson et al. (2005) Cell 122:303-315) and testis (Dym (1994) Proc. Natl. Acad. Sci., USA, 91: 11287-11289; Kobota et al. (2003) Proc. Natl. Acad. Sci., USA, 100: 6487-6492). Comparing with ES cells, tissue-specific stem cells have less self-renewal ability, although they differentiate into multiple lineages. Current evidence indicates that the capability of adult stem cells to give rise to many different specialized cell types is more limited than that of embryonic stem cells (Jiang et al. (2002) Nature 418: 41-49). Therefore, a single adult stem cell has not been shown to have the same degree of pluripotency as embryonic stem cells.

Much excitement has been raised in recent years about the possibility that adult mammalian stem cells may be capable of differentiating across tissue lineage boundaries, and as such may represent novel, accessible, and very versatile effectors of therapeutic tissue regeneration (Wagers and Weissman et al. (2004) Cell 116: 639-648; Quesenberry et al. (2005) Experimental Hematology 33: 389-394). Homeostatic cell replacement and tissue regeneration in the adult dependent on tissue-resident stem cells generated only those mature cell types corresponding to their tissue of origin, and do not cross tissue boundaries to generate cell types of different lineages (Weissman (2000) Cell 100: 157-158). However, recent experiments have challenged this notion and called into question that lineage commitment of various adult stem cell populations by suggesting that under certain circumstances these cells may “transdifferentiate” to contribute to a much wider spectrum of differentiated progeny than previously anticipated (Weimann et al. (2003) Proc. Natl. Acad. Sci., USA, 100: 2088-2093; Bunnell et al. (2005) Can. J. Physiol. Pharmacol 83: 529-539). Transdifferentiation describes the conversion of a cell of one tissue lineage into a cell of an entirely distinct lineage, with concomitant loss of the tissue specific markers and function of the original cell type, and acquisition of markers and function of the transdifferentiated cell type. The suggestion that adult stem cells may transdifferentiate has in turn given rise to the concept of stem cell plasticity, which holds that the lineage determination of a differentiating stem cell may not be rigidly defined, but is instead flexible, allowing these cells to respond to a variety of microenvironmental regenerative cues (Wagers and Weissman (2004) Cell 116: 639-648; Kucia et al. (2005) Leukemia 1-10).

The existence of adult stem cells has been best documented for the hematopoietic system (Weissman (2000) Science 287: 1442-1446). The bone marrow (BM) contains several reconstructing stem cell types, with overlapping phenotypes, including hematopoietic stem cells (HSCs), endothelial stem/progenitor cells (EPCs), mesenchymal stem cells (MSCs), and mutipotent adult progenitor cells (MAPCs). HSCs normally function to generate all of the lineages of mature blood cell types necessary for maintaining proper hematopoietic function (Kondo et al. (2003) Annu. Rev. Immunol., 21: 759-806). The concept that adult HSC function solely to maintain hematopoietic cell lineages was challenged by a series of papers suggesting that unfractionated bone marrow cells, or bone marrow cells enriched by various methods for hematopoietic stem cell activity, could be seen to contribute at low levels to multiple nonhematopoietic tissues following transfer into lethally irradiated, and often injured recipient mice or humans (Herzog et al. (2003) Blood 102: 3483-3493; Goodell (2003) Curr. Opin. Hematol. 10: 208-213). Such studies have reported the expression of donor-derived genetic markers in non-hematopoietic cell within the skin, lung epithelium, intestinal epithelium, kidney epithelium, liver parenchyma, pancreas, skeletal muscle, endothelium, myocardium, and CNS neurons in the cortex and cerebellum (Wagers and Weissman (2004) Cell 116: 639-648; Davani et al. (2005) Cardiovascular Research 65: 305-316) as well as ovary (Johnson et al. (2005) Cell 122:303-15). Such findings were extended by some to a general hypothesis of adult stem cell plasticity, wherein adult stem cells from one tissue were considered to be roughly equivalent in developmental potential to adult stem cells in another tissue, with the outcome of stem cell differentiation largely determined by different microenvironments encountered following differential trafficking from the bloodstream (Blau et al. (2001) Cell 105: 829-841).

BM cell contributions to nonhematopoietic tissues, including myocardium (Orlic et al. (2001) Nature 410: 701-705) and skeletal muscle (Ferrari et al. (1998) Science 279: 1528-1530), also have been reported following direct delivery of cells to injured tissues in unirradiated recipients. The frequency of bone marrow cell contributions nonhematopoietic tissues has varied widely, from less that 0.1% to almost 20% of differentiated cells Goodell (2003) Curr. Opin. Hematol. 10: 208-213; Herzog et al. (2003) Blood 102: 3483-3493). In most cases where BM contributions to nonhematopoietic tissues have been detected, significant tissue injury has been necessary, but some have reported incorporation of cells into tissues without substantial additional injury aside from that induced by the irradiation required for hematopoietic cell transplantation (Krause et al. (2001) Cell 105: 369-377). With a few notable exceptions, in which contribution of transplanted cells to recovery of liver (Lagasse et al. (2000) Nat Med. 6: 1229-1234) or kidney (Kale et al. (2003) J. Clin. Invest., 112: 42-49) function has been documented, most reports of BM or HSC plasticity have to evaluate the tissue-specific function of putatively transdifferentiated cell types. Such determinations clearly will be important in assessing the biological relevance and clinical utility of such events. Disconcertingly, a significant number of studies also report a failure to detect BM and HSC contributions to nonhematopoietic tissues in similar experimental system (Castro et al. (2002) Science 297: 1299; Wagers et al. (2002) Science 297: 2256-225); the reasons for this apparent in ability to reproduce results in different laboratories are not entirely clear, but may relate in part to differences in injury models, detection strategies, identification of donor markers, and/or cell purification techniques (Goodell (2003) Curr. Opin. Hematol. 10: 208-213). Given that in most cases the mechanisms and cell types involved in reported instances of BM or HSC plasticity have not been clearly defined, multiple alternative explanations for such observations remain, and must be evaluated.

Studies of bone marrow stem cell transdifferentiation in adopted tissues have been controversial (Wagers et al. (2004) Cell. 116: 639-48; Vogel (2005) Science 309: 678-679) nevertheless, a growing body of literature suggests that bone marrow stem cells, following transfer into recipient mice can contribute to multiple nonhematopoietic tissues including myocytes, hepatocytes, neurons (Herzog et al. (2003) Blood 102: 3483-3493). Recently, research has demonstrated that bone marrow expresses germline stem cell markers, and bone marrow delivered to the ovaries via the blood stream gives rise to bona fide oocytes in mice (Johnson et al. (2005) Cell, 122: 303-315). However, this possibility has been almost entirely untested in the male.

### SUMMARY OF THE INVENTION

This invention pertains to the discovery that stem cells (e.g., bone marrow stem cells) transplanted directly into a testicular environment are transdifferentiated into bona fide Sertoli cells, and/or Leydig cells, and/or and germ cells. This provides a mechanism for the treatment of male infertility and/or testosterone deficiency.

Thus, in one embodiment, this invention provides a method of treating infertility and/or testosterone deficiency in a male mammal. The method typically involves implanting stem cells into the testes of the mammal whereby the stem cells differentiate into germ cells and/or Sertoli cells and/or Leydig cells thereby reducing infertility and/or testosterone deficiency. In certain embodiments the implanting comprises injecting and/or surgically implanting the stem cells into the testes. In certain embodiments, when injected, the stem cells are injected into the seminiferous tubules of the testes and/or into the interstitium of the testes. In certain embodiments the stem cells differentiate into germ cells and/or Sertoli cells and increase fertility of the male mammal. In certain embodiments the stem cells differentiate into Leydig cells and increase testosterone in the male mammal. The stem cells can be stem cells from the same mammal or from a different mammal (preferably a male mammal) of the same species.

In various embodiments the stem cells can include adult stem cells and/or fetal stem cells, and/or embryonic stem cells, and/or cord blood stem cells and/or amniotic fluid stem cells. The stem cells can be derived from any convenient tissue (e.g. bone marrow). In certain embodiments the stem cells are derived from bone marrow obtained from a bone selected from the group consisting of the hip, the femur, the tibia, the mandible, and the sternum. In certain embodiments the stem cells are in a population of cells comprising non-stem cells. In certain embodiments the stem cells comprise a population of purified stem cells. In certain embodiments the stem cells comprise a population of stem cells expanded ex vivo. In certain embodiments the male mammal is a human (e.g. a human treated with a chemotherapeutic agent, a human subjected to irradiation in the pelvic region, etc.). In certain embodiments the mammal is a non-human mammal (e.g., a horse, a dog, a cat, a sheep, a cow, a pig, a rat, a mouse, a rabbit, a non-human primate, etc.).

This invention also provides a method of inducing the differentiation of stem cells into Sertoli cells and/or Leydig cells and/or germ cells. The method typically involves placing the stem cells in the testes of a male mammal, whereby the stem cells differentiate into germ cells and/or Sertoli cells and/or Leydig cells. In certain embodiments the placing comprises injecting the stem cells and/or surgically implanting the stem cells. The stem cells can be adult stem cells, cord blood stem cells, embryonic stem cells, and the like, e.g. as described above.

Also provided is a composition for the treatment of infertility or testosterone deficiency in a male mammal. The composition comprises stem cells in an excipient acceptable for implantation in the testes of a male mammal. The stem cells can be adult stem cells, cord blood stem cells, embryonic stem cells, and the like, e.g. as described above.

In certain embodiments this invention provides for use of stem cells for the production of a medicament for the treatment of infertility or testosterone deficiency in a male mammal. The stem cells can be adult stem cells, cord blood stem cells, embryonic stem cells, and the like, e.g. as described above.

### DEFINITIONS

The terms “individual” or “subject” refers to a human or an animal subject.

The term “cells” means cells in any form, including but not limited to cells retained in tissue, cell clusters, and individually isolated cells.

The term “non-human mammal” include mammals other than Homo sapiens. Such mammals include, but are not limited to a rodent, largomorph, a bovine, a canine, an equine, a non-human primate, a porcine, and the like).

The phrase “treating infertility or testosterone deficiency in a male mammal” refers to increasing sperm production in said male mammal and/or increasing testosterone production in said male mammal as compared to the same mammal before treatment. In certain embodiments the increase is a measurable increase, preferably a statistically significant increase, e.g. at the 90 percent, preferably at the 95%, and most preferably at the 98 percent or 99 percent confidence level.

### BRIEF DESCRIPTION OF THE DRAWINGS

FIG. 1 shows micrographs of seminiferous tubules observed under florescence microscopy. Seminiferous tubules of a busulfan-treated recipient testis show adult bone marrow-derived cells as GFP-positive (panel A1, fluorescence microscopy; panel A2, transillumination). GFP-positive cells in seminiferous tubules exhibit a spatial and morphological pattern typical for Sertoli cells in recipient testis (panel B1, fluorescence microscopy; panel B2, transillumination). Busulfan-treated wild-type mouse testis without transplantation under fluorescence (panel C1) and transillumination (panel C2) microscopy (negative controls). GFP-positive cells in the interstitium are not readily visible. Scale bar=0.2 mm.

FIG. 2, shows that seminiferous tubules from W/W^{v} recipient testis show adult bone marrow-derived cells as GFP-positive (panel A1, fluorescence microscopy; panel A2, transillumination). GFP-positive cells in seminiferous tubules exhibit a spatial and morphological pattern typical for Sertoli cells in recipient testis (panel B1, fluorescence microscopy; panel B2, transillumination). GFP-positive cells in the interstitium are not readily visible. Scale bar=0.2 mm.

FIG. 3 shows that immunohistochemistry of GFP in recipient testicular sections from busulfan-treated (panel A) and W/W^{v} mice (panel B). GFP staining of germ, Sertoli, and Leydig cells in GFP transgenic mouse testes was used as the positive control (panels C and E). Busulfan-treated mouse testes that did not receive donor cells were used as the negative control (panels D and F). Scale bars: 0.2 mm (panels A-D); 0.05 mm (panels E, F).

FIG. 4 shows immunohistochemistry of GFP in Bouin's fixed, paraffin-embedded testicular sections. GFP-positive Sertoli cell (arrow) in recipient testis was shown in panels A-C. Note of typical morphology of Sertoli cell with both nuclear and cytoplasm staining Panel A: GFP-positive Sertoli cell was detected by using fluorescent secondary antibody. Panel E: GFP-positive preleptotene spermatocytes are shown. Panel F: A clone of germ cells including spermatogonia and spermatocytes was embedded in endogenous spermatogenesis. Panel D: GFP-positive Leydig cells (arrow) were found in the testicular interstitium. Scale bars: 20 μm (panels A, C, F); 50 μm (panels B, D, E).

FIG. 5 shows confocal images showing GFP-positive spermatogonia (panels A, D, and G), VASA expression (panels B, E, and H), and co-localization of GFP with VASA (panels C, F, and I). Asterisks indicate interstitial space. Scale bars=10 μm

FIG. 6 provides confocal images showing GFP-positive donor-derived Sertoli cells (panels A and D), FSH-R expression (panels B and E), and co-localization of GFP with FSH-R (panels C and F). Scale bars=10 μm

FIG. 7 provides confocal images showing GFP-positive donor-derived Leydig cells (panel A), P450scc expression (panel B), and co-localization of GFP with P450scc (panel C). Scale bar=50 μm.

FIG. 8 shows a segment of seminiferous tubule squashed from UBC-GFP transgenic mouse shows GFP-positive cells (panel A) and spermatozoa (panel B) visualized under fluorescent microscope. Note cytoplasmic droplets on spermatozoa (arrows). Scale bars: 0.2 mm (panel A); 10 μm (panel B).

### DETAILED DESCRIPTION

This invention pertains to the surprising discovery that stem cells (e.g., adult stem cells from bone marrow) when transplanted into the testes of a male mammal can differentiate into germ cells, and/or Sertoli cells and/or Leydig cells. In preliminary studies, bone marrow cells harvested from GFP transgenic mice were transplanted directly into the seminiferous tubules and the interstitium of busulfan-treated wild-type mouse testes. The fate of transplanted bone marrow cells in recipient testes was examined 10 weeks after transplantation. Green fluorescence (GFP) positive cells were present both within seminiferous tubules and in the interstitium, indicating that bone marrow cells survive in recipient testes for at least 10 weeks. Immunohistochemistry showed that GFP positive donor cells within the seminiferous tubules were identified as germ cells (preleptotene and pachytene spermatocytes) and Sertoli cells by histologic appearance. GFP positive cells in the interstitium appeared to be Leydig or Leydig-like cells. Some of the GFP-positive Sertoli and Leydig appearing cells expressed androgen receptor (a marker for Sertoli and Leydig cells in the testis), while the germ appearing cells expressed DAZAP1 (a germ cell marker).

These data thus provide evidence showing that adult bone marrow cells once adopted into testicular environment are transdifferentiated into somatic cell as well as germ cell lineages. These data indicate that adult bone marrow (and other stem cell sources) can be a therapeutic source of stem cells that can become spermatogonial cells, Sertoli cells and Leydig cells in a proper testicular microenvironment. This is clinical relevance to the treatment of male infertility and testosterone deficiency.

Thus, for example, the differentiation of stem cells into germ cells offers a treatment for male infertility, e.g. caused by disease, chemotherapy, radiotherapy, and the like. The differentiation of stem cells into Sertoli cells offers a treatment for infertility in those individuals where the problem lies in the Sertoli cells rather than germ cells themselves. In addition, the differentiation of stem cells into Leydig cells offers a treatment for testosterone deficiency or hypogonadism.

In certain embodiments the methods of this invention are particularly useful for the treatment of individuals who have had cancer and irradiation into the pelvic region or chemotherapy. In certain embodiments the methods of this invention can be used as an adjunct or substitute for cryopreservation of germ cells in subjects anticipating radiation into the pelvic region and/or chemotherapy.

In certain embodiments the methods of this invention are also contemplated in certain veterinary applications. Thus, for example, the methods can be used to treat infertility and or testosterone deficiency in horses, cattle, pigs and other non-human mammals.

In various embodiments the methods of this invention involve placing a cell population consisting of or comprising stem cells in the testicular environment (i.e., within the testes). This is readily accomplished, for example, by simply injecting the cell population into the testes. In certain embodiments this entails injecting the cells directly into the seminiferous tubules and/or into the testicular interstitium. In certain embodiments, particularly where differentiation into germ cells is desired, the cell population is injected, e.g., retrograde, into the afferent ducts. Where differentiation into Leydig cells is preferred, injection of the cell population can be preferentially into the interstitium although injection into the seminiferous tubules in this context is suitable as well.

In various embodiments the stem cells will typically be obtained from the same subject to whom they are to be injected. It is contemplated, however, that in certain embodiments the stem cells may be obtained from a different subject of the same species. Thus, in various embodiments, this invention contemplates the use of adult stem cells, and/or embryonic stem cells, and/or core blood stem cells. The cells can be primary cells taken directly from a source and, optionally purified, or, in certain embodiments the stem cells can be expanded in vitro prior to administration to the subject. Where the stem cells are derived from a source other than the subject to whom they will be administered, in certain embodiments the source mammal will be a male mammal.

It is presently believed that stem cells can be found in essentially any mammalian tissue. Thus, stem cells used in the methods of this invention can be obtained from any convenient tissue. In this context, it is noted that embryonic stem cells have been obtained from male testicles (see, e.g., Nature (DOI: 10.1038/nature04697)). Other convenient tissue sources include, but are not limited to bone marrow, fat tissues (see, e.g., U.S. Patent Publications 2005/0282275, 2005/0153442, and 2005/0153441 which are incorporated herein by reference), skin (e.g., hair follicles etc., see, e.g., U.S. Patent Publications 2005/0272147, 2005/0106723, and 2005/0250202 which are incorporated herein by reference), solid tumors (see, e.g., U.S. Patent Publications 2006/0073125 and 2005/0089518 which are incorporated herein by reference), blood, liver, brain/neural tissue (see, e.g., U.S. Patent Publication 2005/0118143 which is incorporated herein by reference), meningeal tissue (see, e.g., U.S. Patent Publication 2004/0014211), teeth or dental pulp (see, e.g., U.S. Patent Publications 2005/0106724 and 2004/0058442 which are incorporated herein by reference), placenta, pancreas (see, e.g., U.S. Patent Publication 2004/0005301 which is incorporated herein by reference), nasal mucosal tissues, and/or muscle tissues, gastrointestinal tissue (see, e.g., U.S. Patent Publication 2005/0256077 which is incorporated herein by reference), amniotic fluid (see, e.g., U.S. Patent Publication 2005/0118712 which is incorporated herein by reference), foreskin (see, e.g., U.S. Patent Publication 2004/0067580 which is incorporated herein by reference), and the like.

In certain embodiments, this invention contemplates the use of genetically engineered stem cells and/or stem cells derived from somatic cell nuclear transfer (SCNT) to introduce particular traits and the like (see, e.g., U.S. Patent Publications 2005/0196858, 2005/0181507, 2005/0170506, 2005/0164385 which is incorporated herein by reference).

While other tissue sources are suitable, bone marrow provides the most convenient source of adult stem cells. Methods of removing bone marrow cells from a subject are well known to those of skill in the art. Bone marrow samples are typically obtained by aspiration through a needle inserted into the bone. In certain embodiments bone marrow aspiration will be done at several places on the body to remove enough bone marrow cells for the transplant to work. Typically, bone marrow is aspirated from the back of the hipbone (posterior superior illium). In certain embodiments the sample can be obtained from the breastbone (sternum), or from the front of the hipbone (anterior iliac crest). In various embodiments the sample can be taken from the front of the lower leg bone (tibia), just below the knee, and so forth.

The bone marrow cells can, optionally, be isolated (e.g. by centrifugation) and resuspended as desired in a suitable buffer/excipient. In certain embodiments this invention contemplates the injection into the testes of a cell population comprising stem cells. The cell population can be a mixed population comprising stem cells as well other cells. Thus, in certain embodiments, a crude or partially fractionated bone marrow (or other source tissue) aspirate/homogenate is used.

In various embodiments, however, the stem cells can be isolated from the source tissue prior to administration to the testes. Methods of isolating adult stem cells from various tissues, core blood stem cells, and embryonic stem cells are well known to those of skill in the art. Thus, for example, Chen et al. (2003) Immunity 19(4): 525-33) teaches the use of a combination of cell surface receptors and intracellular dyes to isolate a stem cell population. In particular, Chen et al. teach that endoglin positive (Endo^{Pos}) and Sca-1 positive (Sca-1^{Pos}) and rhodamine-123 low cells (Endo^{Pos} Sca-1^{Pos} Rh^{Low}) phenotype, without using CD34, c-Kit, or lineage markers, defines a nearly homogenous population of long-term repopulating hematopoietic stem cells (LTR-HSCs).

The isolation of adult stem cells from bone marrow is also taught in U.S. Pat. No. 7,015,037 which is incorporated herein by reference). In the methods described in this patent, bone marrow mononuclear cells were derived from bone marrow aspirates, by standard means known to those of skill in the art (see, e.g., Muschler et al. (1997) J. Bone Joint Surg. Am. 79(11): 1699-709; Batinic et al. (1990) Bone Marrow Transplant, 6(2): 103-107). The multipotent adult stem cells are present within the bone marrow (or other organs such as liver or brain), but do not express the common leukocyte antigen CD45 or erythroblast specific glycophorin-A (Gly-A). The mixed population of cells is subjected to a Ficoll Hypaque separation. Cells are then subjected to negative selection using anti-CD45 and anti-Gly-A antibodies, depleting the population of CD45+ and Gly-A+ cells, and recovering the remaining approximately 0.1% of marrow mononuclear cells. Cells could also be plated in fibronectin coated wells and cultured for 2-4 weeks after which the cells are depleted of CD45+ and Gly-A+ cells. Alternatively, positive selection is used to isolate cells using a combination of cell-specific markers (described in U.S. Pat. No. 7,015,037) such as the leukemia inhibitory factor (LIF) receptors. Both positive and negative selection techniques are known to those of skill in the art, and numerous monoclonal and polyclonal antibodies suitable for negative selection purposes are also known in the art (see, e.g., Leukocyte Typing V, Schlossman, et al., Eds. (1995) Oxford University Press) and are commercially available from a number of sources. Techniques for mammalian cell separation from a mixture of cell populations have also been described in U.S. Pat. No. 5,759,793 (magnetic separation), by Basch, et al. (1983) J. Immunol. Meth. 56: 269 (immunoaffinity chromatography), and by Wysocki and Sato (1978) Proc. Natl. Acad. Sci., USA, 75: 2844 (fluorescence-activated cell sorting).

Recovered CD45−/GlyA− cells are plated onto culture dishes coated with 5-115 ng/ml (preferably about 7-10 ng/ml) serum fibronectin or other appropriate matrix coating. Cells are maintained in Dulbecco Minimal Essential Medium (DMEM) or other appropriate cell culture medium, supplemented with 1-50 ng/ml (preferably about 5-15 ng/ml) platelet-derived growth factor-BB (PDGF-BB), 1-50 ng/ml (preferably about 5-15 ng/ml) epidermal growth factor (EGF), 1-50 ng/ml (preferably about 5-15 ng/ml) insulin-like growth factor (IGF), or 100-10,000 IU (preferably about 1,000 IU) LIF, with 10-10 to 10-8 M dexamethasone or other appropriate steroid, 2-10 μg/ml linoleic acid, and 0.05-0.15 μM ascorbic acid. Other appropriate media include, for example, MCDB, MEM, IMDM, and RPMI. Cells can either be maintained without serum, in the presence of 1-2% fetal calf serum, or, for example, in 1-2% human AB serum or autologous serum.

It was shown in U.S. Pat. No. 7,015,037 that MASCs cultured at low density express the LIF-R, and these cells do not or minimally express CD44 whereas cells cultured at high density, that have characteristics of MSC, loose expression of LIF-R but express CD44. 1-2% CD45-GlyA− cells are CD44− and <0.5% CD45-GlyA− cells are LIF-R+. FACS selected cells were subjected to quantitative RT-PCR (real time PCR) for oct-4 mRNA. oct-4 mRNA levels were 5 fold higher in CD45-GlyA-CD44− and 20-fold higher in CD45-GlyA-LIF-R+ cells than in unsorted CD45-GlyA− cells. Sorted cells were plated in MASC culture with 10 ng/mL EGF, PDGF-BB and LIF. The frequency with which MASC started growing was 30-fold higher in CD45-GlyA-LIF-R+ cells and 3 fold higher in CD45-GlyA-CD44− cells than in unsorted CD45-GlyA− cells. When human cells are re-seeded at <0.5×10³ cells/cm², cultures grow poorly and die. When re-seeded at >10×10³ cells/cm² every 3 days, cells stop proliferating after <30 cell doublings and this also causes loss of differentiation potential. When re-seeded at 2×10² cells/cm² every 3 days, >40 cell doublings can routinely be obtained, and some populations have undergone >70 cell doublings. Cell doubling time was 36-48 h for the initial 20-30 cell doublings. Afterwards cell-doubling time was extended to as much as 60-72 h.

In various embodiments the stem cells comprise multipotent adult progenitor cells (MAPCs), e.g., as described by Schwartz et al. (2002) J. Clin. Invest., 109: 1291-1302, which is incorporated herein by reference for all purposes. Methods of isolating and expanding human (or other mammal) MAPCs and/or mesodermal progenitor cells (MPCs) are well known to those of skill in the art (see, e.g., Reyes et al. (2001) Blood, 98(9): 2615-2625, which is incorporated herein by reference). Thus for example, in the approach taken by Reyes et al., MPCs were selected by depleting bone marrow mononuclear cells from more than human donors of CD45⁺/glycophorin-A (GlyA)⁺ cells. Cells were cultured on fibronectin with epidermal growth factor and platelet-derived growth factor BB and 2% or less fetal calf serum. It was found that CD45⁻ GlyA⁻ cells, or bone marrow mononuclear cells, gave rise to clusters of small adherent cells. Cell-doubling time was 48 to 72 hours, and cells were expanded in culture for more than 60 cell doublings. MPCs were CD34⁻, CD44^{low}, CD45⁻, CD117 (cKit)⁻, class I-HLA⁻, and HLA-DR-. MPCs differentiated into cells of limb-bud mesoderm (osteoblasts, chondrocytes, adipocytes, stroma cells, and skeletal myoblasts) as well as visceral mesoderm (endothelial cells). Retroviral marking definitively showed that single MPCs can differentiate into cells of limb bud and visceral mesoderm.

Methods of isolating and/or expanding adult stem cells are also described in U.S. Pat. Nos. 6,991,897, and in U.S. Patent Publications 2006/0051833, 2006/0014281, 2006/0014280, 2006/0010509, 2006/0051833, 2006/0008902, 2006/0037092, 2005/0283844, 2005/0282275, 2005/0276793, 2005/0272147, 2005/0277190, 2005/0265980, 2005/0260751, 2005/0260748, 2005/0256077, 2005/0250202, 2005/0239897, 2005/0233448, 2005/0233447, 2005/0221487, 2005/0221482, 2005/0221477, 2005/0214873, 2005/0202428, 2005/0181502, 2005/0180958, 2005/0176143, 2005/0176137, 2005/0181504, and 2005/0170502 which are incorporated herein by reference. Similarly methods of isolating and/or expanding embryonic and/or cord blood stem cells are also well known to those of skill in the art (see, e.g., U.S. Patent Publications 2006/0031944, 2006/0040383, 2006/0014279, 2006/0030042, 2006/0030040, 2005/0260747, 2005/0255588, 2005/0244962, 2005/0196859, 2005/0164383, 2005/0164381, 2005/0164377, 2005/0158854, 2005/0124063, 2005/0124003, 2005/0118713, 2005/0158852, and 2005/0037492 which are incorporated herein by reference

The isolated stem cells can be suspended in a suitable buffer/excipient (e.g. a sterile saline buffer) and administered (e.g. injected) into the testes of the recipient subject. Methods of expanding and/or culturing stem cells are also known to those of skill in the art. Thus, for example U.S. patent publication 2006/0051330 (which is incorporated herein by reference) teaches methods for carrying out ex vivo expansion and ex vivo differentiation of multipotent stem cells. According to the methods described in this patent publication ex vivo expansion of stem cells involves the culturing of stem cells in the presence of Flt3 ligand and at least one growth factor from the group consisting of SCF, SCGF, VEGF, bFGF, insulin, NGF and TGF-.beta. In each case, IGF-1 and/or EGF can optionally additionally be used. In various embodiments one of the following combinations is chosen: a) Flt3 ligand and VEGF; b) Flt3 ligand, SCGF and VEGF; c) Flt3 ligand and EGF; d) Flt3 ligand, EGF and bFGF; and e) the growth factors mentioned in a) to d) in combination with IGF-1 and/or EGF. As stated in the patent publication, by using the above-mentioned growth factors it is possible to achieve a more than hundred fold multiplication of the cell counts. Starting out, for example, from only 50 ml of leukapheresis product 1×10⁹ to 1×10¹⁰ multipotent stem cells are produced already after a 14 day culture. Using this method, it is possible to use sources for stem cells (e.g., blood) that obtainable in a simple manner. As stated in the patent publication, the use of Flt3 ligand promotes differentiation in the presence of VEGF and bFGF. Accordingly, in certain embodiments, this combination is avoided if an expansion of multipotent stem cells is to be aimed at exclusively, i.e. without significant differentiation.

While, in certain embodiments, the cell population comprising stem cells is injected into the testes, as described above, methods of administration need not be limited to injection. Any method of placing the stem cells inside the testes can be suitable. Thus, for example, in certain embodiments, the cell population (comprising stem cells) is placed within the testes in a surgical procedure (e.g. a laparoscopic procedure). The cells so placed can be isolated cells, or, in certain embodiments, the cells can be contained in a biocompatible matrix material e.g. to facilitate cell growth and/or proliferation, and/or differentiation, and/or to reduce/prevent immune recognition. Biocompatible matrix materials (e.g., immunoisolatary materials) are well known to those of skill in the art (see, e.g., U.S. Pat. Nos. 6,905,105; 5,874,099; 5,871,767; 5,834,001; and 5,800,829 which are incorporated herein by reference).

In certain embodiments this invention contemplates kits for the practice of the methods described herein. In certain embodiments the kits include a container containing stem cells as described herein. The kits can optionally include a means (e.g. a syringe, an implantable biocompatible matrix, etc.) for placing the stem cells inside the testes. In certain embodiments the kits can optionally include means for extracting bone marrow cells from the subject and/or one or more reagents for purifying/isolating and/or culturing and/or expanding the stem cells ex vivo. The kits typically additionally include instructional materials teaching the use of stem cells to replace Leydig, Sertoli or stem cells in a male mammal. The instructional materials can also teach preferred dosages, modes of administration, counter-indications, and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such information.

The methods and embodiments described above are intended to be illustrative and not limiting. Using the teachings provided herein, other methods and embodiments will be available to those of skill in the art without undue experimentation.

### EXAMPLES

The following examples are offered to illustrate, but not to limit the claimed invention.

### Example 1

### Fate Of Bone Marrow Stem Cells Transplanted Into The Testis

To assess adult stem cell differentiation in the testis, we injected bone marrow cells from adult green fluorescent protein (GFP) transgenic mice into the seminiferous tubules and the testicular interstitium of busulfan-treated wild-type or c-kit mutant (W/W^{v}) mice. Ten to 12 weeks after transplantation, we examined the fate of the transplanted bone marrow cells and found that they survived in recipient testes. In both the busulfan-treated and W/W^{v} mice, some of the GFP-positive donor cells had a Sertoli cell appearance and expressed follicle-stimulating hormone receptor within the seminiferous tubules. In addition, GFP-positive donor cells were found in the interstitium of recipient testes, and they expressed the cytochrome P450 side chain cleavage enzyme (P450scc). In the seminiferous tubules of busulfan-treated mice, GFP-positive donor cells had the appearance of spermatogonia or spermatocytes and expressed VASA. However, this was not found in the seminiferous tubules of W/W^{v} mice. We conclude that adult bone marrow cells, in a favorable testicular environment, differentiate into somatic and germ cell lineages. The resident neighboring cells in the recipient testis may control site-appropriate stem cell differentiation. This clinically relevant finding indicates a treatment of male infertility and testosterone deficiency through the therapeutic use of stem cells.

### Materials and Methods

Animal Preparation

Male wild-type (C57BL/6), green fluorescent protein (GFP) transgenic breeder mice (C57BL/6-Tg-UBC-GFP) and c-kit mutant homozygous (W/W^{v}) mice were purchased from the Jackson Laboratory (Bar Harbor, Me.). Adult GFP transgenic male mice were generated from our colony and used as bone marrow cell donors. These GFP transgenic mice express GFP under the direction of the human ubiquitin C promoter. These mice express GFP in all tissues examined (FIG. 8).We used two kinds of recipient mice: busulfan-treated mice and W/W^{v} mice (homozygous), which both have been used as standardized recipients for germ cell transplantation (Ogawa et al. (1997) Int. J. Dev. Biol., 41: 111-122; Johnston et al. (2001) Endocrinology, 142: 2405-2408). Busulfan is a chemotherapeutic agent that can eliminate spermatogenesis and induce male infertility. For our study, at 4 weeks of age, recipient wild-type mice were given a single dose of busulfan (50 mg/kg body weight) by intraperitoneal injection to destroy endogenous spermatogenesis. Recipients were then used for transplantation 4 weeks after the busulfan injection (Nagano et al. (2003) Biol. Reprod., 68: 2207-2214). We also used recipient W/W mice that have no germ cells as a result of mutations in the c-kit receptor. Animal handling, experimentation, and the bone marrow and testicular tissue harvesting protocol were in accordance with the recommendations of the American Veterinary Medical Association and approved by the Institutional Animal Care and Use Review Committee of Los Angeles Biomedical Research Institute at Harbor-UCLA Medical Center.

Study Design

To determine whether bone marrow stem cells can differentiate into somatic or germinal cells, we inserted bone marrow cells isolated from GFP transgenic mice directly into the seminiferous tubules and interstitial space of recipient testes in groups of eight busulfan-treated wild-type and W/W^{v} mice. Recipients were sacrificed at 10 and 12 weeks after transplantation, and the results were evaluated. One side of the testes was dissected out and decapsulated. Seminiferous tubules were dispersed gently in 2 ml of Hanks' balanced salt solution held in a Petri dish and the live tissue visualized under Zeiss Axioskop 40 fluorescence microscope (Zeiss, Thornwood, NY). The contralateral testis from each animal was fixed in Bouin's solution, embedded in paraffin, and sectioned for immunohistochemistry to detect cells with GFP alone or GFP co-localized with Sertoli, Leydig, and germ cell markers. The Sertoli cell marker used was follicle-stimulating hormone receptor (FSH-R); the Leydig cell marker used was P450scc, and the germ cell marker used was VASA. Co-staining of GFP with various cell markers was detected by double-immunofluorescence technique in combination with confocal laser-scanning microscopy.

Donor Cel Preparation and Transplantation

Donor bone marrow cells were isolated from 6- to 8-weekold GFP transgenic mice by flushing dissected femurs and tibias with phosphate-buffered saline (PBS) (pH 7.4). The cells were pelleted by centrifugation at 600×g for 5 minutes, after which a single cell suspension was obtained at 34° C. by gentle digestion in calcium- and magnesium-free Hanks' balanced salt solution, which contained 0.05 g of collagenase/ml (Life Technologies, Inc., Grand Island, N.Y.), 0.05 mg/ml DNase (Sigma, St. Louis, Mo.), and 0.025% trypsin (Life Technologies, Inc.). After adding trypsin inhibitor (Sigma), centrifuging, and washing with Dulbecco's modified Eagle medium (Invitrogen Corp. Carlsbad, Calif.), the cells were then counted, pelleted by centrifugation at 600×g for 5 minutes, and resuspended in injection media with 0.04% trypan blue stain (Invitrogen Corp.) (Brinster et al. (1994) Proc. Natl. Acad. Sci., USA, 91: 11298-11302; Boettger-Tong (2000) Biol. Reprod., 63: 1185-1191) at a concentration of 5 to 15 million cells/ml.

Microinjection needles were constructed from 20-μl glass micropipettes (catalog 53432-740; VWR, West Chester, Pa.) drawn on a pipette puller (model P-97; Sutter Instruments, Novato, CA). The tip of each pipette was grounded to a sharp beveled point on a microbeveler (model 48000-F; World Precision Instruments, Sarasota, FL). The injection procedure was a modification of the efferent duct injection procedure previously described (Ogawa et al. (1997) Int. J. Dev. Biol., 41: 111-122; Johnston et al. (2001) Endocrinology, 142: 2405-2408). A small incision was made with a sterile 30-gauge needle ˜3 mm from the efferent bundles' junction with the testis. The tip of the injection pipette was inserted into the bundle and then gently pushed toward the rete testis. As the tip entered the area of the rete, 10 μA of the cell suspension was injected under constant pressure. In addition to transplanting the cells into the seminiferous tubules, they were directly injected into the interstitium via rete testis puncture.

Immunohistochemistry for Detecting Bone Marrow-Derived Cells

Immunohistochemistry was performed on Bouin's fixed and paraffin-embedded testicular sections from recipient mice as previously described (Lue et al. (2003) Endocrinology, 144: 3092-1000). Testicular sections were briefly deparaffinized, hydrated by successive series of ethanol, rinsed in distilled water, and then incubated in 2% H₂O₂ to quench endogenous peroxidases. Sections were blocked with 5% normal horse serum for 20 minutes to prevent nonspecific binding of IgG and subsequently incubated with a 1:500 dilution of a monoclonal anti-GFP antibody (sc-9996; Santa Cruz Biotechnology, Santa Cruz, Calif.) (Johnson et al. (2005) Cell, 122: 303-315). Immunoreactivity was detected using biotinylated anti-mouse IgG secondary antibody followed by avidin-biotinylated horseradish peroxidase complex visualized with diaminobenzidine tetrahydrochloride (DAB) as per the manufacturer's instructions (Mouse UniTect ABC immunohistochemistry detection system; Calbiochem, La Jolla, Calif.).Slides were counterstained with hematoxylin and reviewed with a Zeiss Axioskop 40 microscope. Busulfan-treated testes without transplantations and testes from either wild-type or W/W^{v} mice were processed identically as negative controls. Testes from GFP mice were used analogously as a positive control.

Immunofluorescence and Con focal Analysis for Co-Localization of GFP and Cell-Specific Markers in the Testis

Bouin's fixed testicular sections were used for immunohistochemistry to detect cells with co-localized expression of GFP (green, 1:500) and Sertoli, Leydig, or germ cell markers. The Sertoli cell marker used was FSH receptor (FSH-R), the Leydig cell marker used was P450scc, and the germ cell marker used was VASA. We did not find any immunostaining of FSH-R, P450scc, and VASA in isolated bone marrow cells before transplantation. The specificity of the primary antibodies has been previously described (Lo et al. (2004) Endocrinology 145: 4011-4015; Castrillon et al. (2000) Proc. Natl. Acad. Sci., USA, 97: 9585-9590; Baccetti et al. (1998) FASEB J. 12: 1045-1054). After deparaffinization and rehydration, tissue sections were treated with 2% H₂O₂ in PBS for 10 minutes followed by 20 minutes of incubation with blocking serum (5% normal horse serum) at room temperature. After washing the slides three times in PBS (pH 7.4), sections were incubated with a 1:500 dilution of a monoclonal anti-GFP antibody (Santa Cruz Biotechnology) for 1 hour and then incubated with goat anti-mouse Alexa Fluor 488 (green)-labeled secondary antibody (Molecular Probes, Eugene, Oreg.) for 30 minutes. Then the sections were incubated with one of the following antibodies for 1 hour: FSH-R goat polyclonal antibody (1:100; Santa Cruz Biotechnology, Inc.), P450scc rabbit poly-clonal antibody (1:100; Chemicon Inc., Temecula, Calif.), or VASA (DDX4/MVH) rabbit polyclonal antibody (1:100; Abeam Inc., Cambridge, Mass.). The slides were then treated with another fluorescent secondary antibody for 30 minutes at room temperature. Goat anti-rabbit Alexa Fluor 594 (red)-labeled secondary antibody (Molecular Probes) was used for P450scc and VASA; donkey anti-goat Alexa Fluor 594 (red)-labeled secondary antibody (Molecular Probes) was used for FSH-R.Slides were washed and then mounted in Vectashield mounting medium (Vector Laboratories, Inc., Burlingame, Calif.). For negative controls, sections were processed without the primary antibody, and no signals were detected. Confocal imaging was performed using a TCSSP-MP confocal microscope (Leica Corp., Deerfield, Ill.).

TABLE 1
GFP-Positive Bone Marrow-Derived Cells in Recipient Testes

| | Busulfan-treated | |
|---|---|---|
| | mice | W/W^{v} mice |
| Leydig cells/10⁶ μm | 40.2 ± 17.8* | 11.4 ± 1.98 |
| Sertolicells/10⁶ μm | 10.2 ± 1.4* | 4.2 ± 1.0 |
| Germ cells/10⁶ μm | 6.6 ± 1.8 | None |

Values are the mean ± SEM.
*Significant at P < 0.05.
Note:
Germ cells, Sertoli cells, and Leydig cells were characterized based on their morphologic criteria. Germ cells included spermatogonia and spermatocytes

Morphometric Assessment of GFP-Positive Cels in Testes

The method used for germ cell quantitation was similar to that described previously (Lue et al. (2005) Endocrinology, 146: 4148-4154; Lue et al. (2001) Endocrinology, 142: 1461-1470). In brief, testicular sections were examined with an American Optical Microscope (Buffalo, N.Y.) with a ×40 objective and a ×10 eyepiece. A square grid fitted within the eyepiece provided a reference area of 62,500 μm². GFP-positive Leydig cells, Sertoli cells, and germ cells within 40 grids of testicular sections from each animal were counted.

Statistical Analysis

Statistical analyses were performed using the SigmaStat 2.0 program (Jandel, San Rafael, CA). Results were tested for statistical significance using a t-test. Differences were considered significant if P<0.05.

### Results

Bone Marrow-Derived Cells Were Detected in the Live Testicular Tissue 10 to 12 Weeks after Transplantation

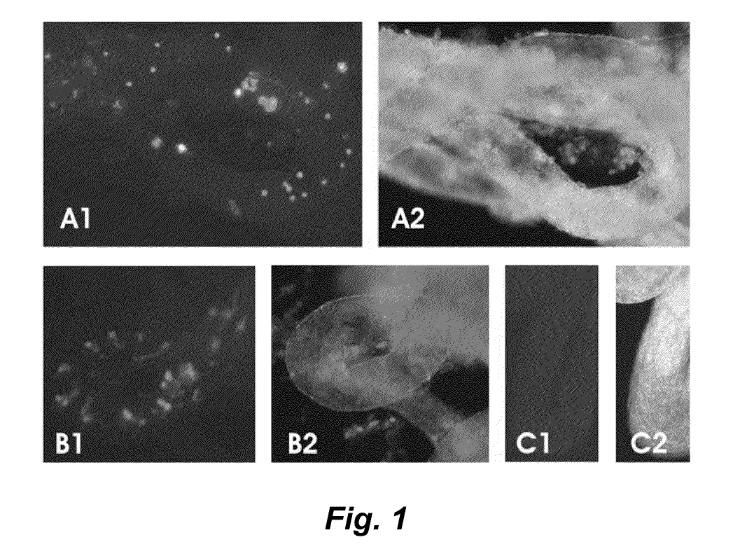

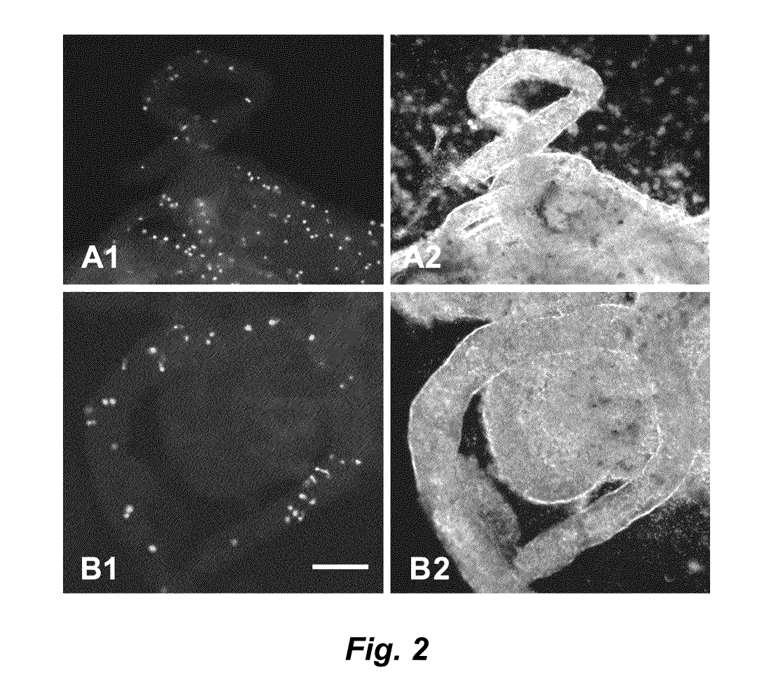

We found that GFP-positive bone marrow-derived cells survived in both busulfan-treated (FIG. 1) and W/W^{v} testes (FIG. 2) for at least 12 weeks after transplantation. GFP-positive cells were observed within seminiferous tubules and in the interstitium in both busulfan-treated (FIG. 1A1) and W/W^{v} (FIG. 2A1) recipient testes. In some of the seminiferous tubules, the green florescent cells extended from the basal lamina toward the luminal compartment and demonstrated a spatial and morphological pattern characteristic of typical Sertoli cells (FIGS. 1B1 and 2B1).

The Bone Marrow-Derived Cells Were Detected as Sertoli, Male Germ, and Leydig Cells by Morphological Assessment and Immunohistochemistry

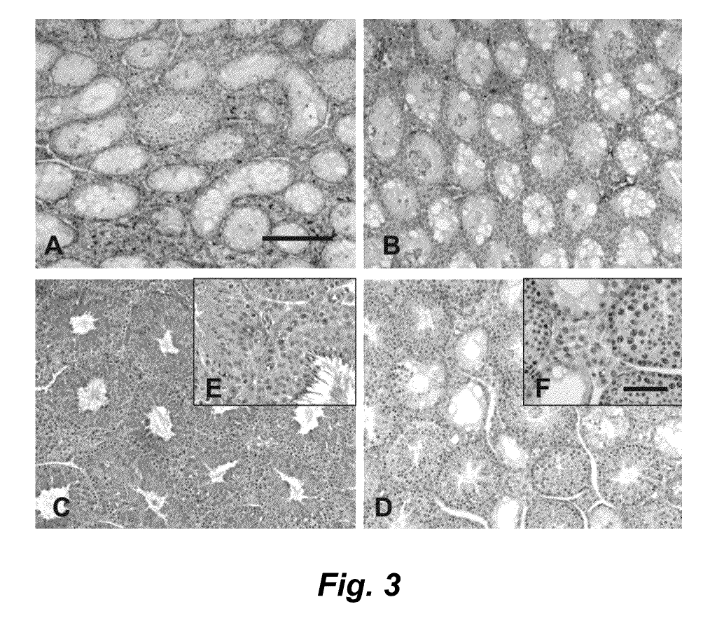

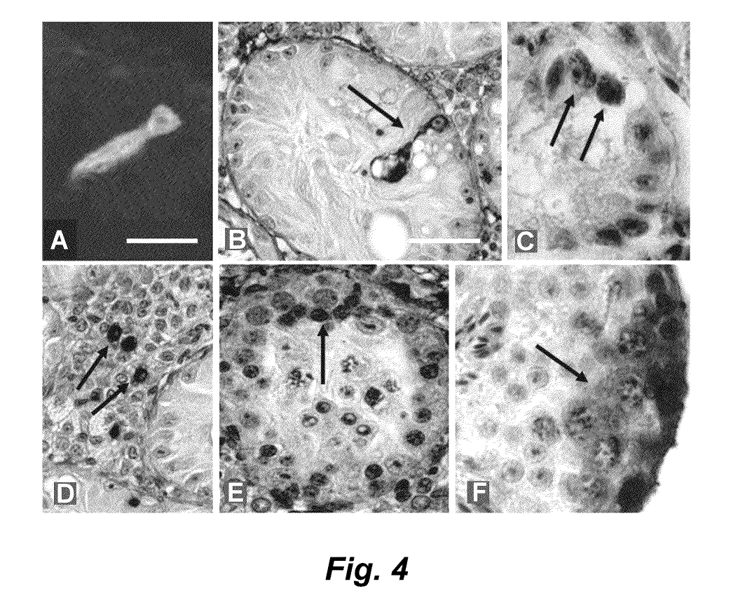

Bone marrow-derived GFP-positive donor cells were further examined by immunohistochemistry and were present in recipient testicular sections from busulfan-treated (FIG. 3A) and W/W^{v} (FIG. 3B) mice. Further morphological examination showed that some of these GFP-positive cells in the busulfan-treated recipient testis had a Sertoli cell appearance (FIG. 4, panels A-C) characterized by an irregular nucleus containing a tripartite nucleolus located near the basal lamina as well as cytoplasm extending from the basal lamina toward luminal compartment. GFP staining was found in both nuclear and cytoplasm of bone marrow cell-derived Sertoli cells. Some of these GFP-positive cells in seminiferous tubules exhibited as a clone consisting of interconnected preleptotene and/or pachytene spermatocytes (FIG. 4, panels E and F). In busulfan-treated mice, the donor-derived germ cells were surrounded and embedded in recovered and endogenous spermatogenesis. No GFP-positive round spermatids were found in the seminiferous tubules of testicular sections examined. In the interstitium, the GFP-positive Leydig cells were readily found embedded in the native Ley-dig cells in the interstitium (FIG. 4, panel D) of busulfantreated and W/W^{v} mice. Quantitative data (Table 1) of GFP-positive Leydig, Sertoli, and germ cells show significantly lower differentiation rates of bone marrow-derived cells in W/W^{v} mice when compared with busulfan-treated mice. Testicular serial sections under confocal microscopy showed that GFP-positive donor-derived Sertoli, Leydig, and germ cells have a single nucleus.

The GFP-Positive Donor-Derived Cells Co-Localized with Cell-Specific Markers in the Testis

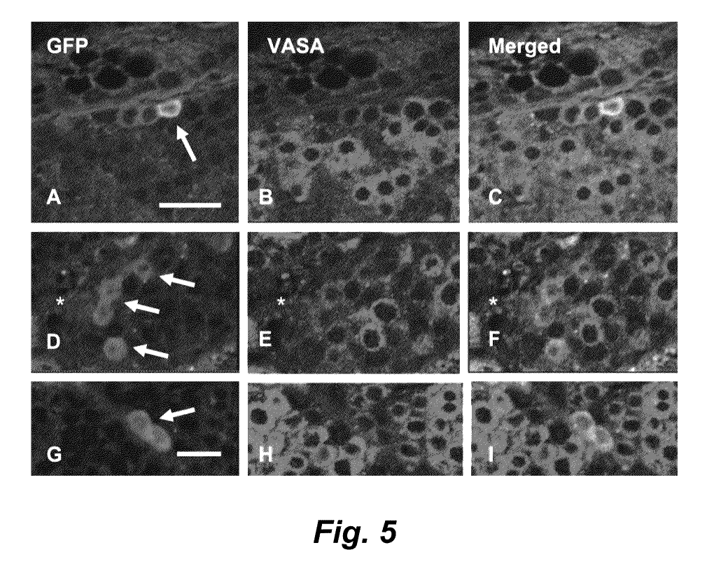

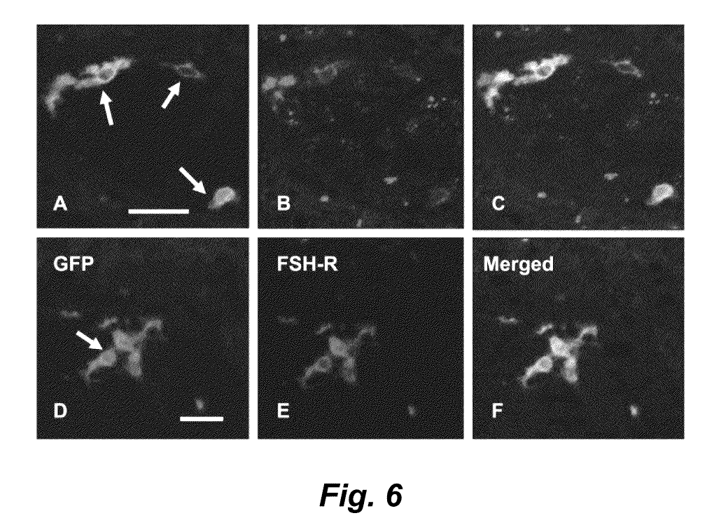

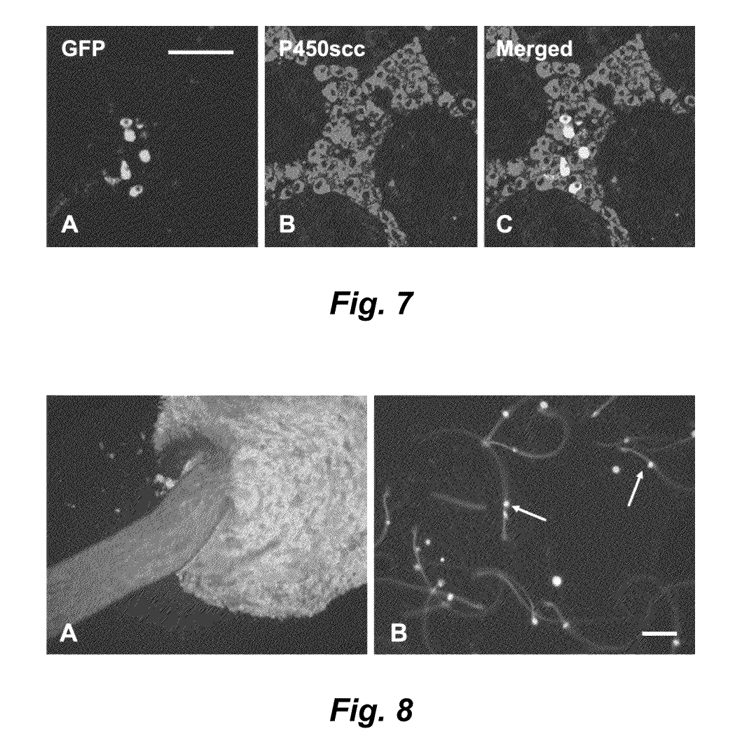

Confocal microscopy demonstrated co-localization of GFP-positive donor-derived germ cells with VASA, a germ cell-specific marker in the testis (FIG. 5). In bone marrow cell-derived germ cells, GFP was expressed in both the cytoplasm and nucleus. VASA protein was detected in both endogenous and donor-derived germ cells in the busulfan-treated recipient testis. Bone marrow cell-derived GFP-positive Sertoli cells expressed FSH-R (FIG. 6). In the interstitium, GFP-positive donor-derived Leydig cells expressed P450scc (FIG. 7), which is a Leydig cell marker in the testis. Twelve weeks after engraftment in W/W^{v} recipient testes, GFP-positive donor cells expressed FSH-R in the seminiferous tubules and P450scc in the interstitium (FIG. 8). However, donor-derived germ cells were not observed in the seminiferous tubules in W/W^{v} recipient testes. A few GFP-positive donor cells with macrophage appearance were occasionally found in the center of the seminiferous tubules in W/W^{v} recipient testes.

### Discussion

We demonstrated that donor-derived GFP-positive cells were present in seminiferous tubules and in the interstitium, indicating that bone marrow-derived cells survive in recipient testes for at least 12 weeks after transplantation. The donor cells used in this study were unfractionated bone marrow cells containing hematopoietic stem cells, endothelial stem/progenitor cells, mesenchymal stem cells, and multipotent adult progenitor cells.

We intended to use busulfan-treated and W/W^{v} mice as our recipients. Busulfan treatment induces chemical injury of spermatogenesis, leading to infertility in male mice. In busulfan-treated recipient testes, we found GFP-positive Sertoli cells, spermatogonia, and early spermatocytes in the seminiferous tubules and Leydig cells in the interstitium. The GFP-positive germ cells were halted at the early spermatocyte stage without further differentiation into spermatids. The mechanisms of donor-derived germ cells that failed to go through meiosis remain unknown. We speculate that donor-derived germ cells arrest at the spermatocyte stage because of their inert genetic imprinting or they are incompatible with the support by Sertoli cells. In the W/W^{v} recipient testes, which are devoid of endogenous germ cells as a result of mutations in the c-kit receptor, we did not find germ cells. A major difference in the two recipient mice is the complete absence of endogenous germ cells in W/W^{v} mice and the presence of spontaneously recovered endogenous germ cells along with donor-derived germ cells in busulfan-treated mice. We found donor-derived germ cells embedded in or surrounded by the spontaneously recovered endogenous germ cells. Donor-derived Sertoli and Leydig cells were also observed among endogenous Sertoli and Leydig cells, respectively. Our observation suggests an essential role of recovering endogenous germ cells in inducing transdifferentiation of donor bone marrow cells into germ cells in the microenvironment of the seminiferous tubules. Endogenous Sertoli and Leydig cells may also play a role in inducing the differentiation of GFP-positive donor-derived Sertoli and Leydig cells because the numbers of GFP-positive cells were significantly higher in busulfan-treated recipient than W/W^{v} mice. Based on this observation, we conclude that on a proper migration of donor stem cells, the resident neighboring cells in the recipient testis may control site-appropriate stem cell differentiation. We found GFP-positive donor-derived cells had a single nucleus in each cell, but we cannot completely exclude the possibility of donor cell fusion with native germ, Sertoli, or Leydig cells.

The percentage of GFP-positive germ, Sertoli, and Leydig cells were low in the recipient testes. The functional status of donor-derived germ, Sertoli, and Leydig cells can be determined. Flow cytometric analysis can be used for quantitative evaluation of donor-derived cells from both interstitial space and seminiferous tubules of recipient testes with cell-specific markers (Kubota et al. (2003) Proc. Natl. Acad. Sci., USA, 100: 6487-6492; Nayernia et al. (2005) Mol. Reprod. Dev., 70: 406-416). To increase the uptake and transdifferentiation of bone marrow cells, cultured and/or isolated and enriched adult stem cells alone and/or with growth factors such as glial cell line-derived neurotrophic factor, stem cell factor, and insulin-like growth factors can be used. Isolation of hematopoietic stem cells has been achieved (Spangrude et al. (1988) Science, 241: 58-62). Clonogenic in vivo and in vitro assays suggest a high level of purity (−85 to 95%) is attainable for these cells (Wagers et al. (2002) Science, 297: 2256-2259). Multipotent marrow stromal cells, which give rise to multiple mesenchymal lineages, can also be isolated from bone marrow (Prockop et al. (2003) Proc. Natl. Acad. Sci., USA, 100: 11917-11923; Gronthos et al. (2003) J. Cell. Sci., 116: 1827-1835).

The molecular mechanism of adult stem cell plasticity is not completely understood. The testis creates a unique microenvironment for donor stem cell migration, proliferation, differentiation, and apoptosis. The testis is protected from immunological influences by the blood-testis barrier allowing the recipient to host donor cells without rejection. By transplanting adult stem cells isolated from gene knockout or transgenic mice into wild-type mice, or vice versa, we are able to study the effect of gene mutations on stem cell biology.

The pathogenesis of male infertility is attributable either to the failure in germ cell proliferation and differentiation or to somatic cell dysfunction. In many cases, germ cells are present. The presence of donor-derived somatic cells is critical because both Leydig and Sertoli cells support spermatogenesis. Defects in these cells have been believed to contribute to abnormal spermatogenesis (Salameh and Swerdloff (2005) pp. 383-416 In: Conditions affecting Sertoli cells. Sertoli Cell Biology. Edited by M K Skinner, M D Griswold. San Diego, Elsevier Academic Press). The possibility of beneficial hormonal effects of Leydig cell transplantation independent of their support of spermatogenesis also exits. Because Leydig cells are responsible for testosterone production, stem cell transplantations can replace the need of life-long testosterone supplementation in male hypogonadism or aging (Swerdloff and Wang (2004) Best Proc. Res. Clin. Endocrinol. Metab., 18: 349-362; Liu et al. (2004) J. Clin. Endocrinol. Metab., 89: 4789-4796). Thus, the present finding has a major impact in understanding reproductive physiology and recovery from testicular pathology and also provides novel therapies in patients with testicular failure.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of treating infertility and/or testosterone deficiency in a male mammal, said method comprising:
implanting stem cells into the testes of said mammal whereby said stem cells differentiate into germ cells and/or Sertoli cells and/or Leydig cells thereby reducing infertility and/or testosterone deficiency.

2. The method of claim 1, wherein said implanting comprises injecting said stem cells into the testes or surgically implanting stem cells.

3. (canceled)

4. The method of claim 2, wherein said injecting comprises injecting said stem cells into the interstitium of the testes or the seminiferous tubules of the testes.

5. (canceled)

6. The method of claim 1, wherein stem cells differentiate into germ cells and/or Sertoli cells and increase fertility of said male mammal, or differentiate into Leydig cells and increase testerone in said male mammal.

7.-8. (canceled)

9. The method of claim 1, wherein said stem cells are selected from the group consisting of adult stem cells, fetal stem cells, embryonic stem cells, multipotent adult progenitor cells, and stem cells derived from somatic cell nuclear transfer.

10.-12. (canceled)

13. The method of claim 1, wherein said stem cells are selected from the group consisting of cord blood stem cells, stem cells derived from bone marrow, and stem cells derived from amniotic fluid.

14.-17. (canceled)

18. The method of claim 1, wherein said stem cells are in a population of cells comprising non-stem cells.

19. The method of claim 1, wherein said stem cells comprise a population of purified stem cells.

20. The method of claim 1, wherein said stem cells comprise a population of stem cells expanded ex vivo.

21. The method of claim 1, wherein said stem cells are from the same mammal or a different mammal of the same species.

22. (canceled)

23. The method of claim 1, wherein said stem cells are derived from the same mammal prior to treatment for cancer and are administered after treatment for cancer.

24.-25. (canceled)

26. The method of claim 1, wherein said mammal is a human treated with a chemotherapeutic agent or subject to irradiation.

27.-29. (canceled)

30. A method of inducing the differentiation of stem cells into Sertoli cells and/or Leydig cells and/or germ cells, said method comprising:
placing said stem cells in the testes of a male mammal, whereby said stem cells differentiate into germ cells and/or Sertoli cells and/or Leydig cells.

31. (canceled)

32. The method of claim 30, wherein said placing comprises injecting said stem cells into the seminiferous tubules of the testes or into the interstitium of the testes, or surgically implanting said stem cells.

33.-34. (canceled)

35. The method of claim 30, wherein said stem cells are from said male mammal.

36. The method of claim 30, wherein said stem cells are selected from the group consisting of adult stem cells, fetal stem cells, embryonic stem cells, multipotent adult progenitor cells, and stem cells derived from somatic cell nuclear transfer.

37-39. (canceled)

40. The method of claim 30, wherein said stem cells are selected from the group consisting of cord blood stem cells, stem cells derived from bone marrow, and stem cells derived from amniotic fluid.

41.-44. (canceled)

45. The method of claim 30, wherein said stem cells are in a population of cells comprising non-stem cells.

46. The method of claim 30, wherein said stem cells comprise a population of purified stem cells.

47. The method of claim 30, wherein said stem cells comprise a population of stem cells expanded ex vivo.

48.-50. (canceled)

51. The method of claim 30, wherein said mammal is a human treated with a chemotherapeutic agent, or subjected to irradiation.

52.-54. (canceled)

55. A composition for the treatment of infertility and/or testosterone deficiency in a male mammal, said composition comprising stem cells in an excipient acceptable for implantation in the testes of a male mammal.

56. The composition of claim 55, wherein said stem cells are selected from the group consisting of adult stem cells, cord blood stem cells, amniotic fluid stem cells, and embryonic stem cells.

57.-58. (canceled)
